(19) United States
(12) Patent Application Publication
(10) Pub. No.: US 2003/0069635 A1
(43) Pub. Date: Apr. 10, 2003

(54) Prosthetic heart valve

(76) Inventors: Richard G. Cartledge, Boca Raton, FL (US); Leonard Lee, New York, NY (US)

Correspondence Address: SMITH, GAMBRELL & RUSSELL, LLP, SUITE 3100, PROMENADE II, 1230 PEACHTREE STREET, N.E., ATLANTA, GA, 30309-3592, US

(21) Appl. No.: 10/157,732

(22) Filed: May 28, 2002

Related U.S. Application Data
(60) Provisional application No. 60/294,042, filed on May 29, 2001.

(51) Int. Cl. A61F 2/24

(52) U.S. Cl. 623/2.13; 623/2.12

(57) ABSTRACT

A novel durable prosthetic heart valve compatible with implantation in a human natural heart valve annulus. The prosthetic heart valve comprises a tubular heart valve which in function resembles a human heart valve, but which is formed of either synthetic or biologic material. The present valve is capable of structurally complying with annular deformation during each heartbeat. Valve embodiments comprise aortic, mitral, tricuspid, and pulmonic implantable valves. Valves can be selectively impregnated with a group of biologically active substances consisting of antibiotics, bactericidal agents, anticoagulant medications, endothelial cells, genetic material, growth factors or other hormonal or biologically active substances. Use of non-thrombogenic biocompatible materials in the valve which mimics operation of a natural heart valve essentially eliminates the need for long term administration of anticoagulants. The current configuration of the valve allows for either percutaneous placement or placement through open techniques.

### RELATED APPLICATION

This application is related to provisional patent application No. 60/294,042 filed May 29, 2001.

### FIELD OF THE INVENTION

This invention relates to implantable heart valves and in particular to long-lasting implantable prosthetic heart valves comprising valve leaflets made from synthetic or biologic materials. The present invention also relates to flexible leaflet heart valves that are used to replace the natural aortic, mitral, tricuspid, or pulmonary valves of the heart. These valves are designed to be placed either percutaneously or by traditional approaches.

### BACKGROUND AND DESCRIPTION OF RELATED ART

A multiplicity of replacement heart valve prostheses are generally known in the art. A first replacement type comprises totally mechanical heart valves which effect unidirectional blood flow through the use of a device using a mechanical closure. Earlier mechanical heart valves comprise pressure responsive, pressure directed movement of a ball in a cage or tilting or caged discs. Other valves known as “tissue valves” utilize either processed cadaveric valves known in the art as homografts, processed and mounted animal valves, or specially prepared and mounted biologic tissues that function as a valve such as bovine pericardial valves.

Examples of pressure responsive, pressure directed ball movement devices are found in U.S. Pat. Nos. 3,263,239, 3,365,728, 3,466,671, 3,509,582, 3,534,410, and 3,723,996. Earliest valve designs were strictly concerned with providing a one-way valve that could be used as a replacement for natural mitral and aortic valves. The earliest known artificial caged ball prosthesis was first successfully used for treatment of cardiac valve disease in 1953. With improvements in valves and medical procedures, caged valve prostheses rapidly became commonplace in the early 1960's.

A source of historical and background information in mechanical valve prostheses is found in The Fourth Edition of Thoracic and Cardiovascular Surgery, published in 1983 by Appleton-Century-Crofts, a publishing division of Prentice-Hall, inc. The earliest caged ball valve comprised a stainless steel outflow orifice and a rib cage and silicone rubber poppets.

Such valves experienced a high incidence of thromboembolism associated with the outflow orifices and rib cages. The silicone rubber poppets after a period of use often became grossly deformed with resulting incompetence. To slow the degeneration of the silicone rubber poppets, cloth and plastic coverings were provided for the metal parts. Such coverings resulted in effects of wear and tissue growth in the coverings. The tissue growth, especially in the coverings over the struts of the cages led to a thickening of the struts that can slow or stop ball movement. Fibrous growth across the orifice of the valve led to severe valvular stenosis.

The use of hollow metal spheres and metal tracks in later models of the caged ball rib valves have overcome some of the original problems, and improvements continue to be made to make caged rib ball valves safer and more effective.

However, problems inherent with the geometry of the caged ball valve also lead to physiologic problems with the use of the valve as a heart valve replacement prosthesis. The caged rib ball valve comprises three orifices through which blood must flow. The primary orifice is the orifice through which blood passes from the effluent chamber being valved. From the primary orifice the blood passes through a secondary orifice defined by the cage and the ball, the size of which is determined by the height of the cage and diameter of the ball. The third orifice is the hollow cylindrical path between the ball and the cage and the surrounding influent chamber into which the blood flows from the effluent chamber.

The three orifice pattern in a caged ball valve requires sometimes difficult tradeoffs to be made in design. For example, when the ball is large, the third orifice is relatively small leading to third orifice stenosis. When the ball is small, the primary orifice is small and relatively stenotic. Further, if travel of the ball in the cage is restricted, as may be required by physiologic free space in either the ascending aorta or left ventricle of a patient, the second orifice size must be reduced with resulting relative stenosis thereat. For these reasons, even in a caged ball valve without physiologic or structural complications, use is restricted by the inherent three orifice geometry.

Disc valves have been made in the form of caged disc valves and tilting disc valves. Disc valves are generally preferred over caged ball valves because of the inherent low profile configuration of the disk. One of the major problems with disc valves and in particular with caged disc valves, is thrombogenicity. Other problems comprise obstructive characteristics inherent to the basic geometry of caged disc valves and degeneration of the disc occluder and strut fracture. Also hemolysis with disc prostheses is especially common.

An example of a tilting disc valve is found in U.S. Pat. No. 4,892,540. Tilting disc valve prostheses have proved to be more satisfactory than the caged disc valves. The tilting disc valve prostheses generally have less hemolysis, lower cross valve gradients, and little wear of carbon pyrolyte discs. However, the tilting disc prostheses have a tendency to clot, and a strict anticoagulant regimen is required. Also movement of the disc in close relation with the sewing ring generally increases chances of interference by contact with adjacent mural endocardium or aortic intima and requires extra care be taken to prevent interference with movement of the disc.

A second replacement type of heart valve prosthesis is the “tissue-type” valve that structurally resembles and functions similarly to at least one of the human heart valves. Such valves are most often harvested from pigs or cows and are mounted on a prosthetic stent with an affiliated sewing ring for attachment to the annulus of the valve being replaced. Problems related to the requirement for anticoagulants are usually short term with “tissue-type” valves and failure of such valves is seldom abrupt.

However, such valves are generally slowly rejected from the patient as a foreign body. The rejection is manifested as motion limiting calcification of the leaflets of the “tissue-type” valve and slowly ensuing functional failure. Such failure commonly necessitates replacement within fifteen years of original implantation. Examples of devices that apply to human and other animal “tissue-type” valvular prostheses are found in U.S. Pat. Nos. 3,656,185 and 4,106,129. Two examples of currently manufactured and marketed “tissue-type” valves are the MITROFLOWTM Heart Valve by Mitroflow International, Inc., 11220 Voyager Way, Unit 1, Richmond, B.C., Canada V6X 351 and Bovine Pericardial Valve by Sorin Biomedical, S.P.A., 13040 Saluggia (VC), Italy.

Prosthetic heart valves comprised of assemblies having various amounts of biological or natural material are often used. As described in more detail below, some of these valves include leaflets derived from natural material (typically porcine) and still include the natural supporting structure or ring of the aortic wall. In other valves, leaflets derived from natural material (typically bovine pericardium) are trimmed and attached to a synthetic, roughly annular structure or ring that mimics the function of the natural aortic wall. In still other valves, both the leaflets and the annular support ring are formed of synthetic polymers or biopolymers (e.g., collagen and/or elastin). For ease of description, these valves will be referred to herein as bioprosthetic valves.

Many bioprosthetic valves include an additional support structure or stent for supporting the leaflets, although so-called stentless valves are also used. The stent provides structural support to the cross-linked valve, and provides a suitable structure for attachment of a sewing cuff to anchor or suture the valve in place in the patient.

The another type of bioprosthetic valve includes individual valve leaflets which are cut from biological material, e.g., bovine pericardium. The individual leaflets are then positioned on the stent in an assembly that approximates the shape and function of an actual valve.

In the case of either type of stented bioprosthetic valve, the function of the stent is similar. Primarily, the function of the stent is to provide a support structure for the prosthetic valve and to maintain the geometry of the valve for proper function. Such a support structure may be required because the surrounding aortic or mitral tissue has been removed in harvesting the valve. The support offered by a stent in a valve is important for several reasons. First of all, a valve is subject to significant hemodynamic pressure during normal operation of the heart. Upon closing the valve the leaflets close to prevent backflow of blood through the valve. In the absence of any support structure, the valve cannot function properly and will be incompetent. One function of the stent is to assist in absorbing the stresses imposed upon the leaflets by this hemodynamic pressure. This is typically achieved in existing stents through the use of commissure support posts to which the valve commissures are attached.

Some known stents have been designed such that the commissure support posts absorb substantially all the stresses placed on the valve by hemodynamic pressure. One such stent is a formed piece of spring wire which is bent to form three vertically-extending commissure support posts, each having a U-shape and being connected to the other commissure support posts via arcuate segments of wire. Such a stent is described in U.S. Pat. No. 4,106,129 to Carpentier, et al. In that stent, the leaflet stresses are home by the commissure posts rotating around and exerting a torque upon the arcuate wire sections between the posts. The composition and structure of this stent also provides for defonnability of the orifice-defining elements. A separate insert element in the form of a plastic web is positioned around the wire stent prior to attachment of the valve.

In other types of stents, the commissure posts are fixed to a rigid base and are designed to be substantially flexible along their entire length so that the posts bend in the manner of a fishing pole in response to the stresses imposed upon the leaflets by hemodynamic pressure. An example of such a stent is shown in U.S. Pat. No. 4,343,048 to Ross, et al.

Other stents, for example the stent shown in U.S. Pat. No. 4,626,255 to Reichart, et al., include further support structure connected to and disposed between the commissure support posts. Such support structure prevents a given commissure post from being resilient along its entire length. Still other stents, such as in U.S. Pat. No. 5,037,434 to Lane, include an inner support frame with commissure posts resilient over their entire length, and a relatively more rigid outer stent support which begins to absorb greater stress as the associated commissure support bends further inward.

Although all of these stents provide support to the bioprosthetic valves to which they are attached, the stress distributions are often unnatural, leading to premature wear or degradation of over-stressed portions of the valve. Accordingly, the need exists for a structure that more closely approximate the stress response of a natural aortic or mitral valve. Stents that include several parts are mechanically complex and require multiple assembly steps.

Another function of a stent is to serve as a framework both for attachment of the valve, and for suturing of the valve into place in the recipient, e.g., a human patient. Toward that end, the stent, or a portion of the stent, is typically covered with a sewable fabric or membrane, and may have an annular sewing ring attached to it. This annular sewing ring serves as an anchor for the sutures used to attach the valve to the patient.

A variety of different stent designs have been employed in an effort to render valve attachment, and implantation of the valve simpler and more efficient. Design trade-offs have often occurred in designing these stents to have the desirable stress and strain characteristics while at the same time having a structure that facilitates assembly and implantation.

Bioprosthetic valves that do not include a stent (“stentless”) are typically of two types. In one type, an actual heart valve is retrieved from either a deceased human (“homograft”) or from a slaughtered pig or other mammal (“xenograft”). In either case, the retrieved valve may be trimmed to remove the aortic root, or the aortic root or similar supporting structure may be retained. The valve is then preserved and/or sterilized. For example, homografts are typically cryopreserved and xenografts are typically cross-linked, typically in a glutaraldehyde solution.

In stentless valves, the unsupported valve is sewn into the recipient's aorta in such a way that the aorta itself helps to absorb the stresses typically absorbed by a stent. Current porcine aortic stentless valves, such as porcine aortic stentless valves, are typically intended for use in the aortic position and not in the mitral position. A mitral valve would require a support structure not presently available with porcine aortic valves, and recently, stentless porcine mitral valves for placement in the mitral position have been developed.

Stented valves used in the mitral position utilize the stent to provide support for normal valve function. In these stented mitral valves, a “low profile” stent having generally shorter commissure posts has been used, so as to prevent the ventricular wall from impinging on the valve. However, use of a lower profile stent often requires that the bioprosthetic valve be somewhat distorted upon attachment to the low-profile stent. This, in turn, can lead to reduced functionality of such valves. While the “higher profile” stents can avoid this distortion, care must be given to valve placement so as to avoid the referenced impingement by the ventricular wall.

Known stents for bioprosthetic valves have been formed from a variety of materials including both metals and polymers. Regardless of the material employed, the long-term fatigue characteristics of the material are of critical importance. Unusually short or uneven wear of a stent material may necessitate early and undesirable replacement of the valve. Other material characteristics are also considered in selecting a stent material, including: rate of water absorption, creep, and resilience to the radiation that may be used for sterilization. Most existing stents are formed of a material having a constant cross-sectional dimension. Formed wire stents and stents fon-ned from stamped metal are examples.

When a patient's own heart valve becomes diseased, it can be either repaired or surgically replaced with an artificial valve. The two basic types of artificial heart valves are mechanical valves and tissue valves. Mechanical valves are made of metal, carbon compounds or hard plastic, whereas tissue valves consist of chemically preserved animal tissue, usually extracted from pig (porcine) or cow (bovine). The animal tissue valves are mounted on a supporting frame or “stent”. The stent enables the surgeon to insert and mount the valve into the heart with minimal difficulty. The stents themselves are constructed from a polymer material and are covered with DACRON.RTM. cloth that contains a sewing ring. Typically, three stent posts project upwardly from the sewing ring and hold the three valve leaflets suspended in the required geometry.

Animal tissue valves have some inherent advantages over mechanical valves since they do not require the patient to be on chronic anticoagulants. Unfortunately, tissue valves eventually suffer from failure in a manner similar to human heart valves, and therefore need periodic replacement. Currently, the survival rate of bioprosthetic tissue valves is approximately 95% after five years from surgery, but only 40% after fifteen years from surgery. The failure of these animal tissue valves results from poor mechanical properties. Specifically, the supporting stents are relatively rigid, and cannot mimic the cyclic expansion and contraction of the natural annulus within which the valve sits. It is believed that mounting of the valves on such non-physiologic stents contributes to mechanical damage caused by repetitive sharp bending at the stent posts. Much of the damage to the valve tissue occurs during valve opening because the supporting stents cannot dilate with the recipient's annulus. Such unnatural behavior induces sharp curvatures within the leaflets and very high local stresses at the hingepoint of the leaflets that damage the leaflet material and ultimately cause it to fail through flexural fatigue.

Another prior art bioprosthetic valve is disclosed in U.S. Pat. No. 5,258,023 (Reger). This valve incorporates a stent comprising a frame that is fully covered by a biocompatible or physiologically compatible shroud. The frame is in the form of a hollow cylinder of rectangular cross-section that is machined or trimmed to provide a suturing support ring, extended cusp stanchions, and interference free blood flow to the coronary arteries. The frame is joint free but is made slightly deformable to conform to contractile changes of the heart. The Reger Patent discloses that such deformity and expansion permits the frame to compliantly respond to expansion and contraction of the native valve orifice of the beating heart in which the aortic valve is implanted in order to reduce beat-by-beat stress on the aortic valve and anchoring sutures, thereby reducing the likelihood of eventual valve failure.

Conventionally, ball or disk valves are used to replace natural mitral, tricuspid, aortic or pulmonary valves of the heart and comprise a rigid frame defining an aperture and a cage enclosing a ball or a disk. When blood flows in the desired direction, the ball or disk lifts away from the frame allowing the blood to flow through the aperture. The ball or disk is restrained by the cage by struts or by a pivot. When blood tries to flow in the reverse direction, the ball or disk becomes seated over the aperture and prevents the flow of blood through the valve. The disadvantage of these valves is that the ball or disk remains in the blood stream when the blood flows in the desired direction, and this causes a disturbance to blood flow.

Flexible leaflet valves mirror natural heart valves more closely. These valves have a generally rigid frame and flexible leaflets attached to this frame. The leaflets are arranged so that, in the closed position, each leaflet contacts its neighbor thereby closing the valve and preventing the flow of blood. In the open position, the leaflets separate from each other, and radially open out towards the inner walls of influent structure. The leaflets are either made from chemically treated animal tissue or polyurethane material. The leaflets must be capable of withstanding a high back pressure across the valve when they are in the closed position, yet must be capable of opening with the minimum pressure across the valve in the forward direction. This is necessary to ensure that the valve continues to correctly operate even when the blood flow is low, and to ensure that the valve opens quickly when blood flows in the desired direction.

A wide range of geometries are used to describe natural aortic valve leaflets during diastole, but these geometries cannot be used for valves made from pericardial or synthetic materials due to the approximately isotropic properties of such materials compared to the highly anisotropic material of the natural valve. Consequently, different geometries have to be used to form flexible leaflet heart valves made from pericardial or synthetic materials with isotropic mechanical properties.

Conventional flexible leaflet heart valves have three substantially identical leaflets mounted onto the frame. The leaflets have a range of designs, both in the geometry of the leaflet and the variations in thickness of the leaflets. Original flexible leaflet heart valves incorporate leaflets that are spherical or conical when in the relaxed state, that is when no pressure is acting on the leaflet. More recently, cylindrical and ellipsoidal leaflets have been proposed. These leaflet geometries are formed with an axis of revolution in a plane generally parallel to the blood flow through the valve.

Prosthetic heart valves are used to replace damaged or diseased heart valves. In vertebrate animals, the heart is a hollow muscular organ having four pumping chambers: the left and right atria and the left and right ventricles, each provided with its own one-way valve. The natural heart valves are identified as the aortic, mitral (or bicuspid), tricuspid and pulmonary valves. Prosthetic heart valves can be used to replace any of these naturally occurring valves. Two primary types of heart valve replacements or prostheses are known. One is a mechanical-type heart valve that uses a pivoting mechanical closure to provide unidirectional blood flow. The other is a tissue-type or “bioprosthetic” valve which is constructed with natural-tissue valve leaflets which function much like a natural human heart valve, imitating the natural action of the flexible heart valve leaflets which seal against each other or coact between adjacent tissue junctions known as commissures. Each type of prosthetic valve has its own attendant advantages and drawbacks.

Operating much like a rigid mechanical check valve, mechanical heart valves are robust and long lived but require that valve implant patients utilize blood thinners for the rest of their lives to prevent clotting. They also generate a clicking noise when the mechanical closure seats against the associated valve structure at each beat of the heart. In contrast, tissue-type valve leaflets are flexible, silent, and do not require the use of blood thinners. However, naturally occurring processes within the human body may attack and stiffen or “calcify” the tissue leaflets of the valve over time, particularly at high-stress areas of the valve such as at the commissure junctions between the valve leaflets and at the peripheral leaflet attachment points or “cusps” at the outer edge of each leaflet. Further, the valves are subject to stresses from constant mechanical operation within the body. Accordingly, the valves wear out over time and need to be replaced. Tissue-type heart valves are also considerably more difficult and time consuming to manufacture.

Though both mechanical-type and tissue-type heart valves must be manufactured to exacting standards and tolerances in order to function for years within the dynamic envirormient of a living patient's heart, mechanical-type replacement valves can be mass produced by utilizing mechanized processes and standardized parts. In contrast, tissue-type prosthetic valves are made by hand by highly trained and skilled assembly workers. Typically, tissue-type prosthetic valves are constructed by sewing two or three flexible natural tissue leaflets to a generally circular supporting wire frame or stent. The wire frame or stent is constructed to provide a dimensionally stable support structure for the valve leaflets that imparts a certain degree of controlled flexibility to reduce stress on the leaflet tissue during valve closure. A biocompatible cloth covering on the wire frame or stent provides sewing attachment points for the leaflet commissures and cusps. Similarly, a cloth covered suture ring can be attached to the wire frame or stent to provide an attachment site for sewing the valve structure in position within the patient's heart during a surgical valve replacement procedure.

With many years of clinical experience supporting their utilization, tissue-type prosthetic heart valves have proven successful. Recently their use has been proposed in conjunction with mechanical artificial hearts and mechanical left ventricular assist devices (LVADS) in order to reduce damage to blood cells and the associated risk of clotting without using blood thinners. Accordingly, a need is developing for a tissue-type prosthetic heart valve that can be adapted for use in conjunction with such mechanical pumping systems. This developing need for adaptability has highlighted one of the drawbacks associated with tissue-type valves-namely, the time consuming and laborious hand-made assembly process. In order to provide consistent, high-quality tissue-type heart valves having stable, functional valve leaflets, highly skilled and highly experienced assembly personnel must meticulously wrap and sew each leaflet and valve component into an approved, dimensionally appropriate valve assembly. Because of variations in tissue thickness, compliance and stitching, each completed valve assembly must be fine-tuned using additional hand-crafted techniques to ensure proper coaptation and functional longevity of the valve leaflets. As a result, new challenges are being placed upon the manufacturers of tissue-type prosthetic valves in order to meet the increasing demand and the increasing range of uses for these invaluable devices.

Accordingly, there is a continuing need for improved prosthetic heart valves which incorporate the lessons learned in clinical experience, particularly the reduction of stress on the valve leaflets while maintaining desirable structural and functional features. Additionally, there is a growing need for improved prosthetic heart valves that can be adapted for use in a variety of positions within the natural heart or in mechanical pumps, such as artificial hearts or ventricular assist devices, as well as alternative locations in the circulatory system. Further, in order to address growing demand for these devices, there is a need for heart valves that are simpler and easier to manufacture in a more consistent manner than are existing valves. Ideally, there is a need for a prosthetic heart valve that is easily and consistently manufactured that obviates the need for chronic anticoagulation with improved longevity beyond that of bioprosthetic replacement heart valves.

### BRIEF SUMMARY AND OBJECTS OF THE INVENTION

In brief summary, the present invention alleviates the known problems related to the substantial and long term requirement for administrating anticoagulants, stenotic operation especially with a low profile valve prosthesis, thrombogenicity, and longevity. The invention is a long-lasting implantable prosthetic heart valve that is made of synthetic or biologic material.

The present heart valve may be produced as a unicast or extruded prosthetic heart valve and is devoid of tilting or traveling metal or plastic components. The invention can be used without a stent or can include a stent that provides an implanting support for the heart valve. The stent may provide a hard surface component to which anchoring sutures are tied and an optionally used soft component against which the anchoring valve receiving orifice of the heart is free to accommodate to changes in cross sectional dimension and contract as the heart beats.

The present inventive heart valve is of a tube configuration wherein the leaflets open up to substantially form a circle with blood flowing therethrough and close up when blood flow reverses. The valve is manufactured of materials that allow it to be compressed into a compact size thus making it amenable to insertion into a catheter allowing for percutaneous placement into the heart. In this embodiment, there is an included self-expanding stent attached to the prosthetic valve annulus that has an apparatus to allow it to be fixed and sealed in the native valve position without the use of sutures. Therefore, the present valve may be inserted in a manner using catheters in addition to being inserted by open chest techniques.

The present inventive heart valve comprises a valve that in form resembles a collapsible tube that functions as a valve. The valve may be a resilient synthetic resinous material part having an outside diameter that is substantially the same size as the annulus of the valve that it is replacing. The present heart valve may be formed by molding or extruding. Because of its tubular structure, the inflow orifice is of low profile. The valve may be comprised of biologic material. The valve may comprise a plurality of cusps which form medially disposed leaflets which coapt upon closure. Force is not localized to one hinge point, but rather widely distributed over a greater portion of the valve. This in turn leads to less localized material wear which can contribute to its longevity. When the synthetic resinous material from which the valve is molded is porous and chemically compatible, it may be selectively complexed and impregnated with antibiotics, bacteriacidal agents, anticoagulant medications, endothelial cells, genetic material, growth factors or other hormonal or biologically active substances. Certain materials used to manufacture the valve may provide a matrix for cellular in growth and therefore further reduce thrombogenecity. Additionally, the valve may be made of a matrix and can function as a cellular scaffold to stimulate cellular in growth including endothelial cells to essentially create a new autologous biologic valve. This matrix may be made of a substance which absorbs over time leaving the patient with only autologous tissue.

The valve may be secured to the native valve annulus with sutures or the valve may have an integrated stent or connector means to secure it in place in the appropriate position in the heart. Accordingly, it is a primary object to provide a prosthetic heart valve having a mean-time-to-failure that is substantially longer than the expected life span of the patient.

It is another primary object to provide such a durable prosthetic heart valve that is simple in construction and low in manufacturing cost. A further object is to provide a prosthetic heart valve configured entirely of biologic, biochemically-inert, or biocompatible materials.

It is another significant object to provide a heart valve that is devoid of adhesives or bonding resins that might be released into the bloodstream of a receiving patient over a period of time.

It is another significant object to provide a prosthetic heart valve comprising a leaflet valve which is similar to a tube in shape and which may be assembled to include a stent which provides mounting support for the valve in a native orifice from which a natural valve remains or has been excised.

It is a key object to provide at least one embodiment of a prosthetic heart valve configured to replace a natural mitral valve.

It is another key object to provide at least one embodiment of a prosthetic heart valve conformably configured to replace a natural aortic valve.

It is another key object to provide at least one embodiment of a prosthetic heart valve conformably configured to replace a natural tricuspid valve.

It is another key object to proved at least one embodiment of a prosthetic heart valve conformably configured to replace a natural pulmonic valve.

It is another significant object to provide the prosthetic valve without a stent that can conform to the natural valve orifice in which the prosthetic valve resides to mimic the changes in natural valve geometry throughout the entire cardiac cycle.

It is another main object to provide a valve which comprises no centrally disposed members during the time the valve is open, thus creating substantially more laminar flow across the valve orifice thereby reducing turbulence which in turn reduces thrombogenecity.

It is another main object to provide a valve comprising members which move toward the outer surface when the valve is coursed with maximum flow thereby providing a valve having a substantially large flow cross section.

It is another main objective to provide a valve that has non-focal areas of stress on the valve leaflets.

It is a principal object to provide a valve whose region that attaches to the annulus is substantially low profile thereby minimizing turbulent blood flow at the valve inflow area thus substantially minimizing thrombogenicity.

It is another notable object to provide a valve which comprises a tube configuration of substantially the same dimensions as a natural heart valve thereby providing a prosthetic valve of relatively low silhouette compared to other prosthetic valves.

It is a principal object to provide a valve that is non-thrombogenic.

It is a principal object to utilize material, whether biologic or synthetic, which is substantially non-thrombogenic.

It is a principal object to provide a valve that causes minimal hemolysis.

These and other objects and features of the present invention will be apparent from the detailed description taken with reference to accompanying drawings.

### BRIEF DESCRIPTION OF THE DRAWINGS

FIG. 1 is a perspective view of one embodiment of the implantable prosthetic heart valve of the present invention comprising a tubular heart valve.

FIG. 2 is an end view of the FIG. I valve shown in the open position.

FIG. 3 is a perspective view of the FIG. 1 valve with a stent.

FIG. 4 is a perspective view of another embodiment of the heart valve of the present invention.

FIG. 5 is a perspective view of the FIG. 4 valve shown in the closed position.

FIG. 6 is a perspective view of another embodiment of the heart valve of the present invention.

FIG. 7 is a rear perspective view of the FIG. 6 heart valve.

FIG. 8 is a perspective view of another embodiment of the prosthetic heart valve of the present invention shown in the closed position.

FIG. 9 is a perspective view of the FIG. 8 heart valve shown in the open position.

FIG. 10 is a sectional view of a heart showing the prosthetic valve of the present invention in the heart, replacing an aortic valve.

FIG. 11 is a sectional view showing the prosthetic valve of the present invention being inserted by percutaneous placement.

### DETAILED DESCRIPTION OF THE PREFERRED EMBODIMENTS

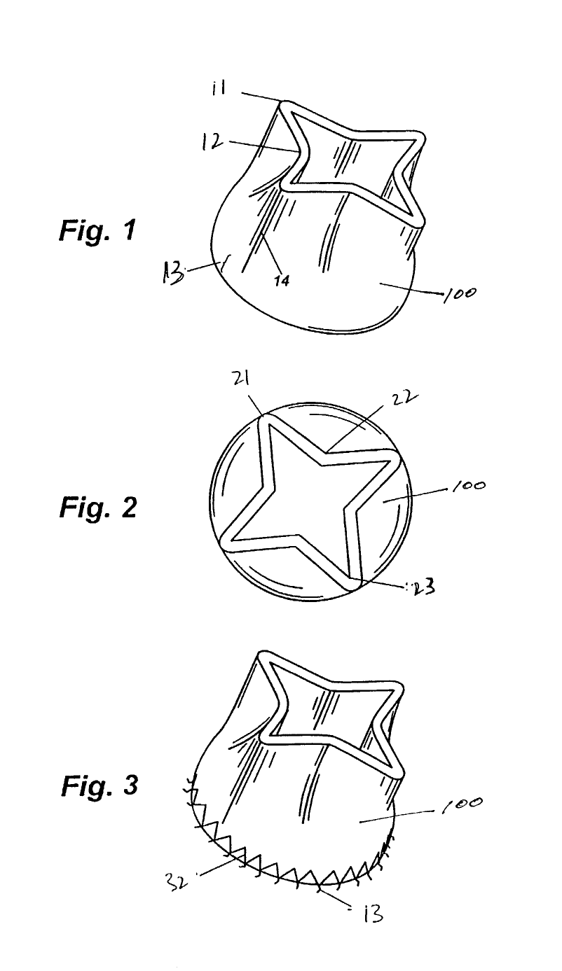

Reference is now made to the embodiments illustrated in FIGS. 1-9.

A first illustrated embodiment of the invention, seen in FIG. 1, is a prosthetic heart valve. Heart valve 100 comprises a heart valve leaflet formed by flexible junctures such as outer crease 11 and inner crease 12. Heart valve 100 includes tubular annulus portion 13 and optional support spars 14 which provide support for flexible juncture outer crease 11 to maintain the valve in position and insure that it does not collapse.

Further, as shown in FIG. 2, heart valve 100 may include support spars 21, 23 at outer crease juncture 11 to provide further support. As shown in FIG. 2 juncture or inner crease 22 may not have such supports so as to facilitate opening and closing at flexible juncture 22.

The valve of the present invention may contain a stent as illustrated in the FIG. 3 embodiment where stent 32 is integrated into the valve at the valve annulus 13.

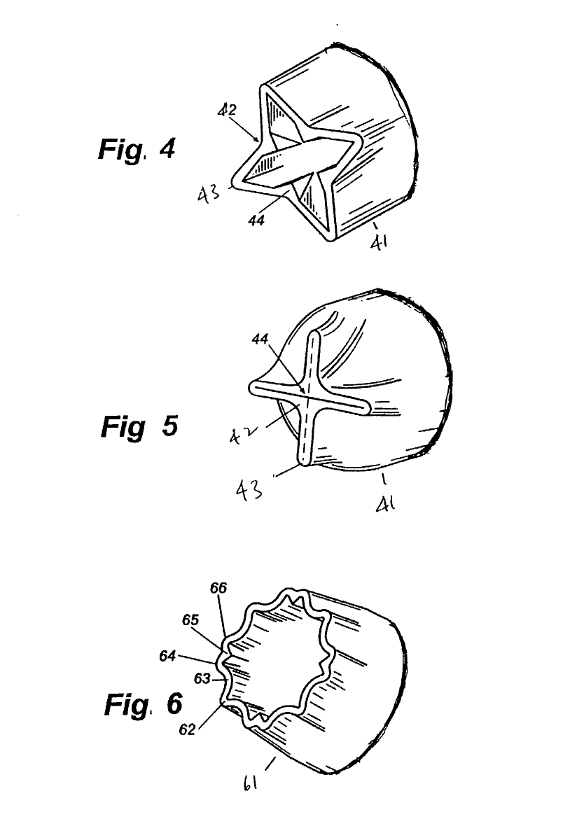

FIG. 4 depicts another embodiment of the present invention wherein tubular valve 41 is in the open position and has flexible junctures or creases 42, 43, and extended portion 44. FIG. 5 shows the FIG. 4 embodiment in the closed position depicting flexible junctures 43 having sufficient support to maintain their relative position while junctures 42 and 43 have flexibility such as to close, thereby shutting off blood flow.

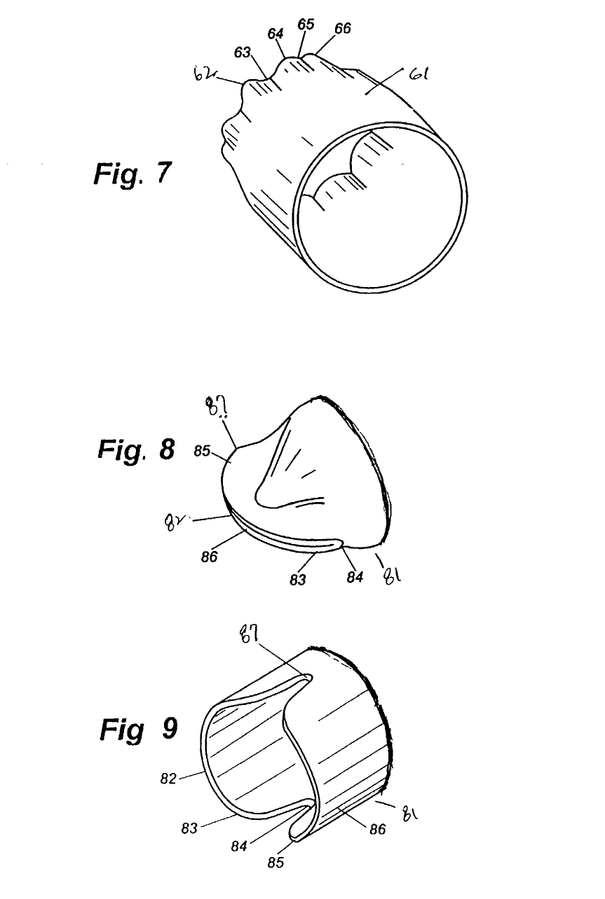

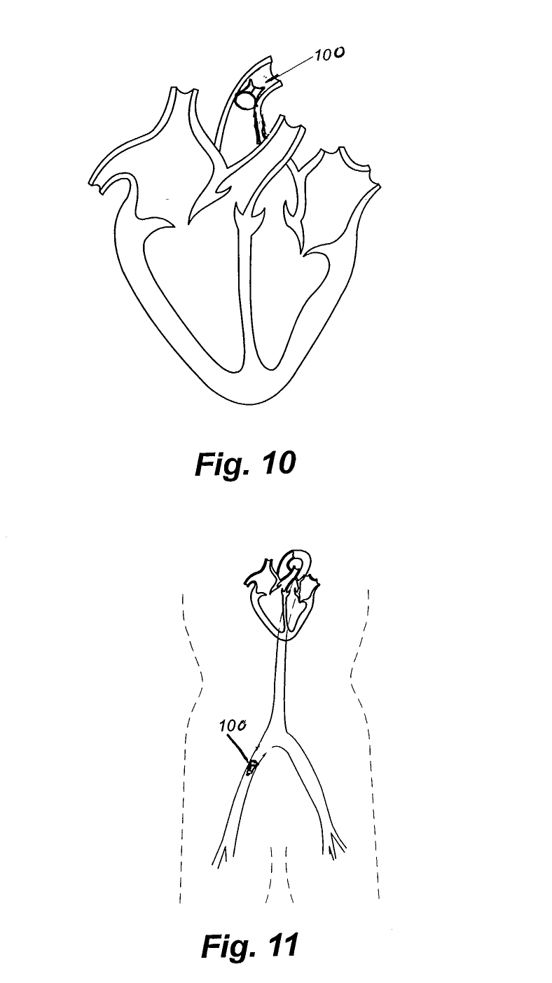

FIG. 6 depicts a further embodiment of the present invention wherein tubular valve 61 contains various flexible junctures or creases 62, 63, 64, 65, and 66. FIG. 7 is a rear perspective view of the valve of the present invention shown in FIG. 6.

FIG. 8 is a further embodiment of the present invention depicting closed tubular valve 81 having flexible junctures or creases 82, 83, 84, 85, 86, and 87. FIG. 9 is a perspective view of the valve depicted in FIG. 8 in the open position.

The materials of construction of the present inventive heart valve must be biocompatible and blo-compatible and have the property of returning to its original, formed shape, i.e. a shaped memory, such that the valves depicted in FIGS. 5 and 8 are in their as formed, closed position and will remain in that position until forced open by the pressure of blood as shown in FIGS. 1, 2, 3, 4, 6, 7 and 9.

The present inventive heart valve is not limited to stentless valves, but may be used in association with a stent. In the case of percutaneous placement, there is included a stent integrated into the prosthetic valve annulus. The stent has structural memory allowing for the entire valve/stent complex to collapse and thus be inserted into a sheath allowing percutaneous placement. Once the valve is deployed into position, the stent expands to its original shape providing a seal and retention into the native valve annulus. The tubular shape is advantageously formed of a single material which is molded or extruded such that the valve, or leaflets, open up to substantially form a circle when blood flows there through and, because of the shaped memory, close up when the blood flow reverses. The material used to form the present heart valve must be a malleable material, i.e. it must allow the tubular valve to be compressed into a sheath and inserted percutaneously using catheters to a position of a native heart valve whose function it replaces. This allows the tubular valve of the present invention to be inserted by percutaneous technique using catheters. The valve material of the present invention has memory and once it is placed with a catheter in the position of the old heart valve, a sheath is pulled back and the valve expands and nests over the old valve. Where an expandable stent is used over the new valve the stent expands up against the old valve and the new valve nests against the annulus of the old valve. Alternatively, a self expanding sheath, similar to a straw with a stent and a new valve may be used to place the new valve.

Preferably the new valve and stent are inserted by collapsing against a balloon, slowly inflating the balloon and through the use of a dye can be correctly placed. Alternatively a stent may be used which has barbs to locate it in the annulus of the old valve. Traditional open chest surgery can also be used to sew the new valve in place after removing the old valve. Coronary arteries come off the aorta, and during the diastolic mode do not completely open, therefore, the valve of the present invention may be formed with an inward camber such that only a portion of the valve, or its leaflets, cover the coronary artery, i.e. such that it will have an inward camber at the position of the left and right coronary arteries.

The valve of the present invention overcomes all problems associated with mechanical heart valves. The significant amount of hemolysis of red blood cells common with metal heart valves is not encountered when using the present inventive valve. The use of potent anticoagulants which is required by many patients using prior art metal heart valves is not needed with the present inventive valve. Because of the material used and the motion of the present inventive valve it will function for the life of the patient rather than having to be replaced after a number of years as must presently be done with tissue (pig) valves. Since there is no defined area of stress or focal point of stress the present tube-like heart valve does not restrict blood flow, nor is it detrimental to red blood cells.

Because of the tubular form and the method of opening and closing the present inventive valve provides lamellar flow and therefore does not obstruct the normal flow of blood and there are no sites for clots to form, thereby lessening or obviating the need for anticoagulants. The present valve not only overcomes the problem with prior art mechanical valves of the hemolysis of red cells being crushed between the surfaces during the mechanical valve closure, but also eliminates the pressure gradient created by prior art valves between the left ventricle and aorta due to the outflow obstruction created by the ball or flap or other projections in the center of flap-type valves. Such a pressure gradient can also cause blood turbulence that can initiate or heighten clotting and other undesirable effects.

As shown in FIG. I an optional support spar 14 may be impregnated or placed in the material to prevent the valve from collapsing during diastolic. Annulus 13 may be in opposition with the aorta or existing, native valve annulus. Inner crease 12 and outer crease II form the bends that enhance the opening and closing of valve 100.

FIG. 2 shows a cross-sectional view of the valve shown in FIG. I wherein support spar 23, inner crease 22 and outer crease 21 are shown.

FIG. 3 depicts an inner vascular deployment embodiment of the valve with an optional stent 31 in the annulus attached to the valve by hooks 32. FIG. 4 depicts valve 41 in the open position having inner crease 42, outer crease 43, and extended portion 44. FIG. 5 depicts valve 41 of FIG. 4 in the closed position.

FIG. 6 depicts valve 61 in the open position having outer creases 62, 64, and 66 and inner creases 63 and 65 that facilitate the opening and closing of valve 61. FIG. 7 is a rear view of valve 61 in the open position. FIG. 8 is a view of tubular valve 81 in the closed position having slots 84 and 87 formed therein, shoulders 83 and 85, and major portions 82 and 86. In the closed position shoulders 83 and 85 are adjacent as are major portions 82 and 86. Also in the closed positions notches 84 and 87 are closed. FIG. 9 depicts valve 81 in the open position in a substantially circular form wherein notches 84 and 87 are open and major portions 86, 82 and shoulders 83, 85 are not adjacent or touching.

While attachment of the heart valve of the present invention is described to be accomplished by sewing, one skilled in the art understands that other methods of attachment, including such as by a plastic-like connector or by fusing parts together are within the scope of the invention. One important factor in the selection of materials for the valve of the present invention is the choice of materials that may be complexed with appropriate biochemicals from a group comprising antibiotics, anticoagulant medications, endothelial cells or endothelial cell growth factors.

Incorporation of complexed antibiotics about the site of the insertion or excision may significantly reduce the risk of post placement infection, potentially reducing the amount of otherwise administered antibiotics and relieving the valve recipient of a post placement antibiotics regimen. Incorporating complexed anticoagulants when possible, in prosthetic valve 100 reduces or eliminates the need for an initial exogenous anticoagulation regimen on the part of the valve recipient. Such a regimen is currently common place for prosthetic heart valve recipients.

In a natural aortic heart valve, the cusps are individually identifiable as a right coronary cusp, a left coronary cusp, and a non-coronary cusp. In the present prosthetic valve, each leaflet cusp may be substantially like the others. For this reason the present invention is described in detail with the understanding that a like description applies for all cusps as well.

As seen in FIG. 1, crease or juncture 1I comprises a thickened superior edge that forms a juncture commissure with more flexible or thinner crease or juncture 12. In this embodiment, the junctures are molded as a unitary part of heart valve 100. The thickening of juncture 11 is necessary to provide reliable structure where flexing and wear is the greatest in heart valve 100.

While it is within the scope of the invention to provide a prosthetic valve having creases or junctures that are manufactured separately and later affixed to each other, the present heart valve 100 preferably is cast as a unit or unicast. Methods for casting such a valve are well known in the art. One material that may be used is silicone. An advantage derived from the use of silicone is the opportunity for complexing with other materials, such as antibiotics to potentially decrease the risk of post placement valve infection and anticoagulant medication to potentially reduce the risk of thrombogenesis.

Heart valve 100 comprises a tubular portion that may be put in place with or without a stent. Where a stent is used the tubular end is affixed to the stent by suturing to the annulus of the native valve.

One material from which heart valve 100 may be made is a synthetic, pliable polytetrafluoroethylene (PTFE) material known as GORTEXTMSURGICAL MEMBRANE. GORTEX SURGICAL MEMBRANE is essentially biocompatible, hydrophobic and nonthrombogenic. It has been used in pleural, peritoneal and pericardial reconstruction.

The present invention may be embodied in other specific forms without departing from the spirit or essential characteristics thereof The present embodiments are therefore to be considered in all respects as illustrative and not restrictive, the scope of the invention being indicated by the appended claims rather than by the foregoing description, and all changes which come within the meaning and range of equivalency of the claims are therefore intended to be embraced therein.

What is claimed is:

1. A durable, bio-compatible prosthetic heart valve which is used as a replacement for a natural heart valve and which is permissive to liquid flow in a first direction and occlusive to liquid flow in the opposite direction, said prosthetic heart valve comprising:
valve means consisting essentially of non-thrombogenic biocompatible material, said valve means being of substantially tubular configuration having at least two leaflets on one end thereof which in their manufactured, normal position are substantially closed and which open up to substantially form a circle allowing blood to flow therethrough when a certain blood pressure level is attained, return to the substantially closed position when the blood pressure decreases to below said certain level, and upon a reversal in blood flow return to the closed position to prevent retrograde blood flow across or through the valve.

2. The prosthetic heart valve of claim 1 which in its manufactured position is completely closed.

3. The prosthetic heart valve of claim 1 which in its manufactured position is partially closed.

4. The prosthetic heart valve of claim 1 wherein the tubular valve comprises surfaces which are functionally contiguous and non-thrombogenic.

5. The prosthetic heart valve of claim 1 manufactured as a unit.

6. The prosthetic heart valve of claim 1 manufactured in separate components and joined together to form one contiguous structure prior to insertion.

7. The prosthetic heart valve of claim 6 further comprising means for adjoining unitable parts of the prosthetic heart valve by stitching or other connecting means void of toxic resins or adhesives which could release into the blood stream of a patient over a period of time.

8. The prosthetic heart valve of claim 1 used as a replacement valve for a natural heart valve that has not been excised from its natural orifice.

9. The prosthetic heart valve of claim 1 used as a replacement valve for a natural valve that has been excised from its natural orifice.

10. The prosthetic heart valve of claim 1 wherein the essentially biocompatible material of the valve is combined with a biologically active substance selected from the group consisting of antibiotics, bactericidal agents, anticoagulant medications, endothelial cells, genetic material, growth factors, other hormonal or biologically active substances, and combinations thereof.

11. The prosthetic heart valve of claim 1 manufactured of a matrix that stimulates cellular in growth.

12. The prosthetic heart valve of claim 11 manufactured of a matrix that is absorbed over time.

13. The prosthetic heart valve of claim 1 wherein the essentially biocompatible material comprises gortex surgical membrane.

14. The prosthetic heart valve of claim 1 wherein said tubular valve consists essentially of silicon.

15. The prosthetic heart valve of claim 1 wherein said tubular valve consists essentially of flexible resilient synthetic resinous material.

16. The prosthetic heart valve of claim 1 wherein said tubular valve consists essentially of PTFE.

17. The prosthetic heart valve of claim 1 wherein said tubular valve consists essentially of polyethylene glycol terephtalate.

18. The prosthetic heart valve of claim 1 wherein said tubular valve consists essentially of biologic materials.

19. The prosthetic heart valve of claim 1 wherein said tubular valve consists essentially of mammalian pericardium.

20. The prosthetic heart valve of claim 1 wherein said tubular valve consists essentially of mammalian tissue lined by endothelium.

21. The prosthetic heart valve of claim 1 wherein said tubular valve consists essentially of biologically engineered tissue.

22. The prosthetic heart valve of claim 1 wherein said valve is inserted by utilizing a catheter for percutaneous placement.

23. The prosthetic heart valve of claim 1 wherein said valve includes a stent.

24. The prosthetic heart valve of claim 1 wherein said valve is inserted by open chest techniques and the natural valve has been excised from its natural orifice.

25. The prosthetic heart valve of claim 1 extruded as a single unit.

26. The prosthetic heart valve of claim 1 extruded as separate components which are joined prior to insertion.

27. The prosthetic heart valve of claim 1 manufactured in the closed or partially closed position wherein said material has a shaped-memory and opens in response to a set blood pressure level and will close in response to a decrease in blood pressure to below said set level or a reversal of blood flow.

28. The prosthetic heart valve of claim 1 which is entirely collapsible and amenable to insertion into a catheter allowing for percutaneous insertion.

29. The prosthetic heart valve of claim 1 which is substantially a collapsible, shaped memory, tube which opens up in response to a set blood pressure.

30. The prosthetic heart valve of claim 1 wherein said tubular valve has an outside diameter substantially equal to the natural valve being replaced.

31. The prosthetic heart valve of claim 1 having no centrally disposed members when the valve is in the open position.

32. The prosthetic heart valve of claim 1 comprising members that move toward the outer surface of its tubular shape when the valve is coursed with antegrade blood flow.

33. The prosthetic heart valve of claim 1 which, when in the open position, provides a substantially laminar blood flow.

34. The prosthetic heart valve of claim 1 which substantially minimizes the pressure gradient across the valve.

35. The prosthetic heart valve of claim 1 wherein said tubular valve has an annulus portion and at least two outer crease flexible junctures.

36. The prosthetic heart valve of claim 35 including supports at said outer crease flexible junctures.

37. The prosthetic heart valve of claim 36 including inner creases between the at least two outer crease flexible junctures, which inner creases do not contain supports.

38. The prosthetic heart valve of claim 1 wherein the valve material has memory, is placed with a catheter in the position of the natural heart valve, without the natural heart valve being removed, and upon placement in the position of the natural heart valve expands and is retained over the natural heart valve.

39. The prosthetic heart valve of claim 38 wherein the prosthetic heart valve is used as an aortic valve replacement and wherein during antegrade flow said valve does not occlude the coronary artery orifices.

40. The prosthetic heart valve of claim 1 which during its operation will not substantially hemolyze red blood cells.

41. The prosthetic heart valve of claim 1 wherein the tubular valve includes means for joining the prosthetic valve to the native valve annulus.

42. The prosthetic heart valve of claim 41 joined to the native valve annulus by means selected from the group consisting of sutures, stents, glues, other adhesives, other connecting means, and combinations thereof.

43. The prosthetic heart valve of claim 1 including rigid or semi-rigid structural supports to add rigidity to certain areas of the valve where rigidity is advantageous.

44. The prosthetic heart valve of claim 10 wherein said biologically active substance is impregnated into said valve material.
